(19) United States
(12) Patent Application Publication
(10) Pub. No.: US 2022/0099582 A1
(43) Pub. Date: Mar. 31, 2022

(54) SYSTEM AND METHOD FOR OPTIMIZING GAS REACTIONS

(71) Applicant: THERMO ENVIRONMENTAL INSTRUMENTS LLC, Franklin, MA (US)

(72) Inventors: Robert Bailey, Bellingham, MA (US); Bryan Marcotte, Blackstone, MA (US); Jeffrey Socha, Boylston, MA (US); Nathan Taylor, Woonsocket, RI (US)

(21) Appl. No.: 17/449,332

(22) Filed: Sep. 29, 2021

Related U.S. Application Data
(60) Provisional application No. 63/085,252, filed on Sep. 30, 2020.

(51) Int. Cl. G01N 21/76; B01L 1/02; B01L 5/00

(52) U.S. Cl. CPC G01N 21/766; B01L 1/02; B01L 2300/0663; B01L 2300/168; B01L 2300/0848; B01L 5/00

(57) ABSTRACT

An embodiment of an analyzer is described that comprises a first conduit configured to channel an annular flow of a first gas; a second conduit positioned within the first conduit, where the outer dimension of the second conduit is separated from an inner dimension of the first conduit by a gap configured to channel an axial flow of a second gas; a reaction chamber fluidically coupled to the first conduit and the second conduit, where the reaction chamber comprises a window on a side opposite from an orifice of the first conduit into the reaction chamber; and a detector positioned adjacent to a side of the window opposite from the reaction chamber, wherein the detector is configured to receive light produced from a reaction of the first gas and the second gas in the reaction chamber.

### FIELD OF THE INVENTION

The present invention is generally directed to a reaction chamber having two gas channels positioned to maximize detection of a signal produced by the mixing of two gasses.

### BACKGROUND

It is generally appreciated that embodiments of chemiluminescence and fluorescence analyzers configured to detect the reactions produced by mixing gasses have been described. A number of such systems have taken various approaches to improving signal detection taking into account the fast kinetics of the reactions.

However, the solutions of the previously described embodiments have imposed additional constraints on the design of the chemiluminescence and fluorescence analyzers and, importantly, lack the ability to make adjustments to control the position of the reaction in the reaction chamber to account for variability.

Therefore, a need exists for a chemiluminescence and fluorescence analyzers configured to maximize detection of a signal produced by the mixing of two gasses with features that enable adjustment of the configuration.

### SUMMARY

Systems, methods, and products to address these and other needs are described herein with respect to illustrative, non-limiting, implementations. Various alternatives, modifications and equivalents are possible.

An embodiment of an analyzer is described that comprises a first conduit configured to channel an annular flow of a first gas; a second conduit positioned within the first conduit, where the outer dimension of the second conduit is separated from an inner dimension of the first conduit by a gap configured to channel an axial flow of a second gas; a reaction chamber fluidically coupled to the first conduit and the second conduit, where the reaction chamber comprises a window on a side opposite from an orifice of the first conduit into the reaction chamber; and a detector positioned adjacent to a side of the window opposite from the reaction chamber, wherein the detector is configured to receive light produced from a reaction of the first gas and the second gas in the reaction chamber.

In some embodiments an orifice of the second conduit is positioned a distance away from the orifice of the first conduit. In some cases, the orifice of the second conduit is positioned in the first conduit to form a mixing region in the first conduit. More specifically the distance from the orifice of the first conduit to the orifice of the second conduit may include a distance in a range of about −0.40″ to about +0.10″, and even more specifically may include a distance of about −0.15″. Also, in some instances a position of the orifice of the second conduit is adjustable relative to the orifice of the first conduit into the reaction chamber.

In some cases, the first gas is O₃ and the second gas is a sample gas that may include NO. In the same or alternative implantations, an internal surface of the reaction chamber with the entrance is substantially parabolic or substantially hemispheric. Further, in some instances the internal surface of the reaction chamber may be substantially reflective.

Further, the orifice of the second conduit may include a nozzle that can be configured as a flared nozzle or as a tapered nozzle. Also, the gap may include a space separation in a range of about 0.005″ to about 0.056″.

A embodiment of a method is also described that comprises (a) channeling an annular flow of a first gas through a first conduit; (b) channeling an axial flow of a second gas through a second conduit positioned within the first conduit, where the outer dimension of the second conduit is separated from an inner dimension of the first channel by a gap; (c) reacting the first gas with the second gas to produce light in a reaction chamber fluidically coupled to the first conduit and the second conduit, where the reaction chamber comprises a window on a side opposite from an orifice of the first conduit into the reaction chamber; and a detecting the light produced using a detector positioned adjacent to a side of the window opposite from the reaction chamber.

In some embodiments an orifice of the second conduit is positioned in the first conduit a distance away from the orifice of the first conduit into the reaction chamber to form a mixing region in the first conduit. In some cases, the orifice of the second conduit is positioned in the first conduit to form a mixing region in the first conduit. More specifically the distance from the orifice of the first conduit to the orifice of the second conduit may include a distance in a range of about −0.40″ to about +0.10″, and even more specifically may include a distance of about −0.15″. Also, in some instances a position of the orifice of the second conduit is adjustable relative to the orifice of the first conduit into the reaction chamber. In some cases, the method may further comprise (d) adjusting a position of an orifice of the second conduit relative to the orifice of the first conduit; and (e) repeating steps (a)-(d) until the position of the orifice of the second conduit produces a maximal value of the light detected from the reaction of the first gas with the second gas.

In some cases, the first gas is 03 and the second gas is a sample gas that may include NO. In the same or alternative implantations, an internal surface of the reaction chamber with the entrance is substantially parabolic or substantially hemispheric. Further, in some instances the internal surface of the reaction chamber may be substantially reflective.

Further, the orifice of the second conduit may include a nozzle that can be configured as a flared nozzle or as a tapered nozzle. Also, the gap may include a space separation in a range of about 0.005″ to about 0.056″.

The above embodiments and implementations are not necessarily inclusive or exclusive of each other and may be combined in any manner that is non-conflicting and otherwise possible, whether they are presented in association with a same, or a different, embodiment or implementation. The description of one embodiment or implementation is not intended to be limiting with respect to other embodiments and/or implementations. Also, any one or more function, step, operation, or technique described elsewhere in this specification may, in alternative implementations, be combined with any one or more function, step, operation, or technique described in the summary. Thus, the above embodiment and implementations are illustrative rather than limiting.

### BRIEF DESCRIPTION OF THE DRAWINGS

The above and further features will be more clearly appreciated from the following detailed description when taken in conjunction with the accompanying drawings. In the drawings, like reference numerals indicate like structures, elements, or method steps and the leftmost digit of a reference numeral indicates the number of the figure in which the references element first appears (for example, element 110 appears first in FIG. 1). All of these conventions, however, are intended to be typical or illustrative, rather than limiting.

FIG. 1 is a functional block diagram of one embodiment of an air monitor in communication with a computer;

FIG. 2 is a simplified graphical representation of one embodiment of an analyzer with a reaction chamber;

FIG. 3 is a simplified graphical representation of a magnified view of one embodiment of the reaction chamber of FIG. 2 with a detector window;

FIG. 4 is a simplified graphical representation of a magnified view of one embodiment of the reaction chamber, and detector window of FIG. 3 with a gas mixing area for a reaction that produces light; and

FIG. 5 is a simplified graphical representation of one embodiment of gas concentration data detected from the light of FIG. 4.

Like reference numerals refer to corresponding parts throughout the several views of the drawings.

### DETAILED DESCRIPTION OF EMBODIMENTS

As will be described in greater detail below, embodiments of the described invention include an analyzer with a reaction chamber having two gas channels positioned to maximize detection of a signal produced by the mixing of two gasses. More specifically, the position at least one of the gas channels is adjustable to control the position of the gas reaction in the reaction chamber.

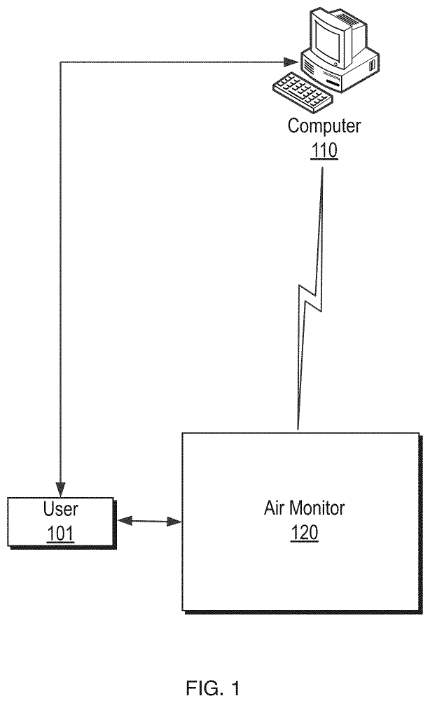

FIG. 1 provides a simplified illustrative example of user 101 capable of interacting with computer 110 and air monitor 120. Embodiments of air monitor 120 may include a variety of commercially available air monitors. For example, air monitor 120 may include the iQ series of gas analyzer instruments available from Thermo Fisher Scientific. FIG. 1 also illustrates a network connection between computer 110 and air monitor 120, however it will be appreciated that FIG. 1 is intended to be exemplary and additional or fewer network connections may be included. Further, the network connection between the elements may include “direct” wired or wireless data transmission (e.g. as represented by the lightning bolt) as well as “indirect” communication via other devices (e.g. switches, routers, controllers, computers, etc.) and therefore the example of FIG. 1 should not be considered as limiting.

Computer 110 may include any type of computing platform such as a workstation, a personal computer, a tablet, a “smart phone”, one or more servers, compute cluster (local or remote), or any other present or future computer or cluster of computers. Computers typically include known components such as one or more processors, an operating system, system memory, memory storage devices, input-output controllers, input-output devices, and display devices. It will also be appreciated that more than one implementation of computer 110 may be used to carry out various operations in different embodiments, and thus the representation of computer 110 in FIG. 1 should not be considered as limiting.

In some embodiments, computer 110 may employ a computer program product comprising a computer usable medium having control logic (e.g. computer software program, including program code) stored therein. The control logic, when executed by a processor, causes the processor to perform some or all of the functions described herein. In other embodiments, some functions are implemented primarily in hardware using, for example, a hardware state machine. Implementation of the hardware state machine so as to perform the functions described herein will be apparent to those skilled in the relevant arts. Also in the same or other embodiments, computer 110 may employ an internet client that may include specialized software applications enabled to access remote information via a network. A network may include one or more of the many types of networks well known to those of ordinary skill in the art. For example, a network may include a local or wide area network that may employ what is commonly referred to as a TCP/IP protocol suite to communicate. A network may include a worldwide system of interconnected computer networks that is commonly referred to as the internet, or could also include various intranet architectures. Those of ordinary skill in the related art will also appreciate that some users in networked environments may prefer to employ what are generally referred to as “firewalls” (also sometimes referred to as Packet Filters, or Border Protection Devices) to control information traffic to and from hardware and/or software systems. For example, firewalls may comprise hardware or software elements or some combination thereof and are typically designed to enforce security policies put in place by users, such as for instance network administrators, etc.

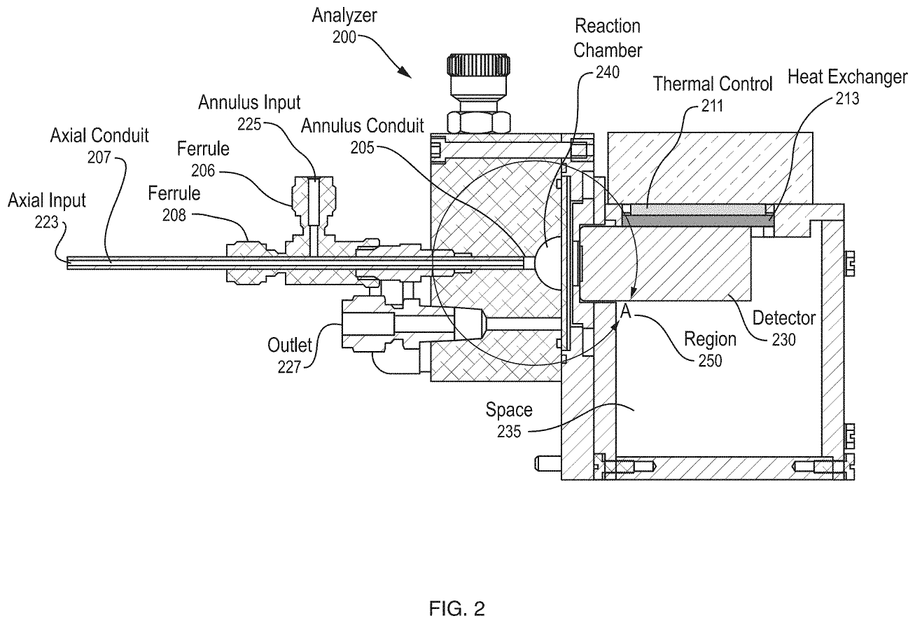

FIG. 2 provides an illustrative example of analyzer 200 that is a component of air monitor 120. As illustrated in FIG. 2, analyzer 200 includes reaction chamber 240, provides sufficient space for a reaction of gasses to occur releasing light and includes an interior surface that is substantially reflective at the wavelengths of light produced by the reaction. Importantly, the dimensions of reaction chamber 240 are configured so that the gasses can substantially react and exit the space as well as maximize the efficiency of light collection. For example, the interior surface of reaction chamber 240 is configured in a substantially hemispheric or parabolic shape that redirects photons of light to a path towards detector 230. In the described embodiments, the substantially hemispheric shape may be configured to provide reflected light that is substantially collimated, whereas the substantially parabolic shape may be configured to provide reflect light with a broad dispersion pattern. In the presently described example, the interior surface of reaction chamber 240 may be coated with a substantially reflective material that is resistant to corrosion and degradation which could result from the gasses used for the reaction. The reflective material may include a chrome material, a gold material, or other suitable material known in the art. The interior surface of reaction chamber 240 may also be polished and/or have other surface finish that improve reflectivity and/or corrosion resistance. In some embodiments reaction chamber 240 includes a polished, gold-plated surface on the substantially hemispheric or parabolic wall that is resistant to corrosion under the reaction conditions, as well as providing beneficial reflection characteristics particularly at the wavelengths of interest.

In the described embodiments, detector 230 may include a Photomultiplier Tube (PMT), photodiode, CCD camera, or other type of detector known in the art. The example of FIG. 2 also illustrates elements configured to regulate the temperature of detector 230 that include heat exchanger 213, thermal control 211, and insulated space 235. For example, in some embodiments variations in temperature of the PMT can result in the introduction of noise in the output signals, and in some embodiments a PMT may have a higher sensitivity at a “cool” temperature. Therefore, in the described example, it may be desirable to maintain the temperature at a substantially constant temperature, which may in some cases follow recommendations by the manufacturer of the PMT that outlines the relationship of dark current to temperature.

Heat exchanger 213 may include a heat sink or any other element known to transfer heat and thermal control 211 may include a thermoelectric heating/cooling device enabled to maintain detector 230 at a desired temperature. Further space 235 may be filled with insulation which further limits temperature fluctuation and/or temperature influences from the ambient environment outside of analyzer 200.

In the same or alternative example, detector 230 may typically be configured with a “wide” field of view, but this configuration is generally quite expensive. However, embodiments of the presently described invention may provide significant advantages in the efficiency of signal detection that may enable the use of less costly implementations of detector 203 that have a narrower field of view. Also, the spectral range of the light produced from the reaction may include a range from about 600 nm to about 3000 nm, where detector 230 may only need to be sensitive to a sub-range to produce accurate results. For example, detector 230 may include a PMT that have a detection range for light in a range from about 230 nm to 920 nm.

FIG. 2 also illustrates annulus conduit 205 that is fluidically coupled with annulus input 225. Axial conduit 207 is positioned within annulus conduit 205 and is fluidically coupled to axial input 223. In the example of FIG. 2, annulus input 225 may fluidically couple to another element of air monitor 120, such as an ozone generator, using ferrule 206. For example, an ozone generator configured for use with the described invention may produce a flow of ozone of about 30-50 mg/hr.

Axial input 223 may similarly couple to another element of air monitor 120, such as a source of calibration gas (e.g. NO, NO₂, etc.) and/or may couple with a source of a sample gas (e.g. ambient air, emissions source such as a smokestack, etc.) using ferrule 208. Importantly, axial conduit 207 may be positionally adjusted within annulus conduit 205 by loosening ferrule 208 and moving axial conduit 207 linearly along the axis of annulus conduit 205. Once a desired position of axial conduit 207 has been attained, ferrule 208 may be tightened to hold axial conduit 207 in that position. In the embodiments described herein, ferrule 206 and ferrule 208 may be constructed from any desirable material known in the art, where some materials may have desirable characteristics over others. For example, ferrules made from a Teflon material do not typically provide a permanent compression but are easier to use for adjustments, whereas ferrules made from a stainless-steel material are desirable for more permanent locking.

It will be appreciated by those of ordinary skill in the art that other clamping mechanisms are known in the art that may be employed in place of ferrules 206 and 208, and thus the examples of ferrules 206 and 208 should not be considered as limiting. Further, annulus input 225 could alternatively couple to the described source of calibration gas and/or source of a sample gas, and that axial input 223 could couple to an ozone generator or another element of air monitor 120.

FIG. 2 also illustrates outlet 227 that is fluidically coupled to reaction chamber 240 and configured to exhaust gasses from reaction chamber 240. Outlet 227 may be coupled to an element of air monitor 120 such as a vacuum pump that may be desirable if reduced pressures within reaction chamber 240 are beneficial for certain applications. For example, in some applications it may be desirable that reaction chamber 240 has a pressure of about 200 mm mercury.

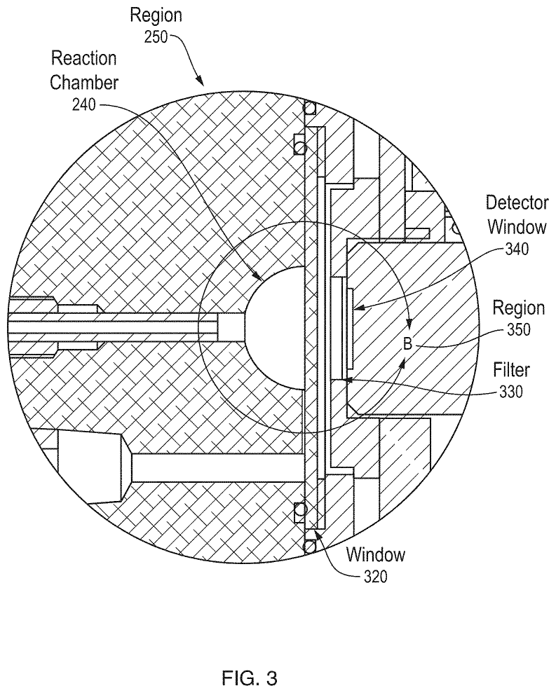

FIG. 2 illustrates region 250 which is magnified in in FIG. 3. FIG. 3 provides an illustrative example of window 320 that separates reaction chamber 240 from detector window 340. It is generally desirable that window 320 can withstand the reaction conditions and environment within reaction chamber 240 to maintain optical transparency for the wavelengths of interest and may be constructed from a quartz material, or other desirable material known in the art. Similarly, it is desirable that window 340 is constructed to protect the elements of detector 230 and maintains optical transparency for the wavelengths of interest. FIG. 3 also illustrates filter 330 that may include what is referred to as a bandpass filter, notch filter, or other type of optical filter known in the art. For example, filter 330 may be configured to reject wavelengths of light that typically produce noise in a detected signal and transmit wavelengths of light associated with a true signal indicating gas concentration to detector window 340.

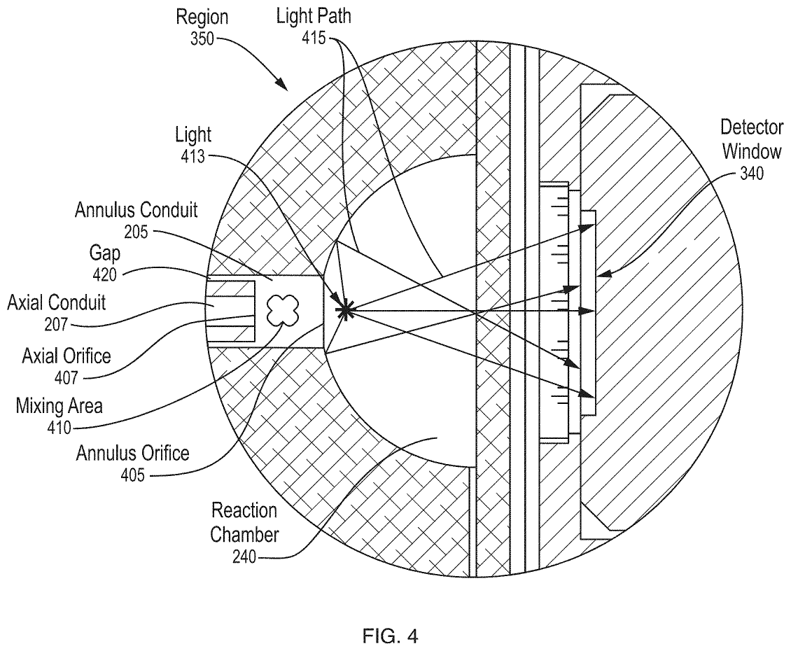

FIG. 3 illustrates region 350 which is further magnified in FIG. 4. FIG. 4 provides an illustrative example of gap 420 that is a space between the outer dimension of axial conduit 207 and the inner dimension of annulus conduit 205. In the described embodiments, gap 420 provides space for the annular flow of gas within annulus conduit 205 (e.g. a ring of flow of gas, such as ozone, around axial conduit 207). For example, annulus conduit 205 may include a cross sectional area of about 0.00361² and the flow may include a rate of about 250 cc/min of gas.

Axial conduit 207 includes an internal channel for the axial flow of another gas (e.g. a calibration gas and/or sample gas) that exits at axial orifice 407 into mixing area 410. For example, axial conduit 207 may include a cross sectional area of about 0.00306² and the flow may include a rate of about 100 cc/min of gas.

The space separation of gap 420 may include a distance in a range of about 0.005″ to about 0.056″. For example, gap 420 may include a distance of about 0.008″. Further, it will be appreciated that the ratio of a cross-sectional area for annulus conduit 205 (e.g. inner diameter) to the cross-sectional area for axial conduit 207 (e.g. outside diameter) may include a range of about 1:1 to up to about 10:1.

In some embodiments, it may be desirable that axial orifice 407 is configured as a tip or nozzle comprising a shape that affects one or more of the characteristics of the flow of the exiting gas. In some embodiments, the tip or nozzle is fitted to the end of axial conduit 207 and may be interchangeable. It will be appreciated that the geometry of the shape of the tip or nozzle and resulting characteristics of the flow has an influence on the kinetics of the reaction. For example, in one embodiment the shape geometry of the tip or nozzle may include what is referred to as a “flare” geometry that influences the exiting gas into a substantially turbulent flow pattern that may promote active mixing of the gasses from orifice 407 and orifice 405 at closer position to axial orifice 407. Alternatively, the shape geometry of the tip or nozzle may include what is referred to as a “taper” geometry that influences the exiting gas into a substantially laminar flow pattern that may delay active mixing the gasses from orifice 407 and orifice 405 to a more distant position from axial orifice 407.

In the described embodiments, the gasses exiting from annulus conduit 205 via gap 420 and axial conduit 207 via axial orifice 407 combine in mixing area 410 and produce a reaction that generates one or more photons of light 413. In some cases, the ratio of gas composition in mixing area 410 is about a 50:50 mix (e.g. ozone and NO), however other ratios may also be used. Those of skill in the art appreciate that the kinetics of the reaction may vary due to one or more conditions that include, but are not limited to, flow rates of the gasses, reaction time of the gasses, temperature, and pressure within reaction chamber 240. The kinetics of the reaction may have influence on the timing of the reaction and/or the position where the reaction takes place in the reaction chamber. It may be generally desirable that the reaction occurs at a position in the reaction chamber were the transmission of light is most efficient (e.g. least amount of light lost that does not reach detector window 340). For example, light path 415 illustrates examples of optical paths where light 413 travels directly to detector window 340, or reflects once off the interior surface of reaction chamber 240 and travels to detector window 340 (e.g. only a single reflection to limit the loss of light 413).

As described above, axial path conduit 207 may be positionally adjusted within annulus path conduit 205 to control the position where reaction between the gasses occur. In the described embodiments, it is highly desirable to be able to control the distance between annulus path orifice 405 and axial path orifice 407 that dictates the position of mixing area 410. It will be appreciated that the kinetics of gas reactions to produce light can be very fast and may depend on variables of the environment within reaction chamber 240 that may be changed (e.g. environmental conditions may include temperature, pressure, etc.). Therefore, it is generally desirable that the position of mixing area 410 is adjustable during initial setup/manufacture, as well as adjustable by the user to compensate for changes in the process flow dynamics. Further, one of more characteristics of reaction chamber 240 may change over time such as, for example, corrosion and/or degradation of the reflective surface on the interior of reaction chamber 240. Therefore, the ability to adjust the position of the reaction within reaction chamber 240 provides a significant advantage to maximize detection efficiency.

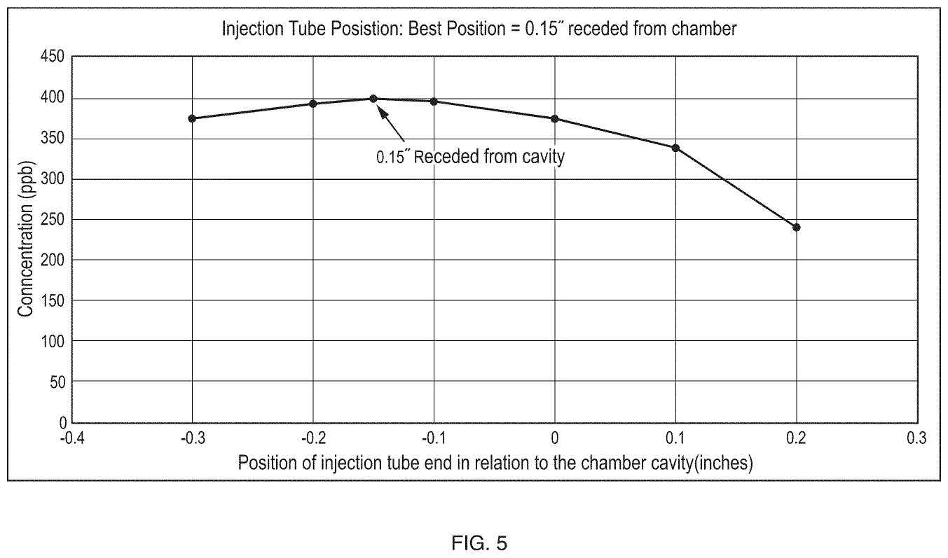

FIG. 5 provides an illustrative example of the differences in detection efficiency of a chemiluminescence reaction using a known concertation of a test gas (e.g. a calibration gas) in reaction chamber 240 with axial orifice 407 of axial conduit 207 at different distances from annulus orifice 405 of annulus conduit 205 into reaction chamber 240. In the described example, the chemiluminescent reaction includes:

NO+O₃→NO₂+O₂+hv

where, NO is the test gas and hv represents the infrared light emission that results when NO2 molecules decay to lower energy states as measured by a PMT detector.

As described above, the distance between orifices 405 and 407 define, in part, the volume of mixing area 410, and in combination with the environmental conditions the position where the reaction of NO and O₃ produces light 413 in reaction chamber 240. In the example of FIG. 5 a test gas of about 400 ppb NO was tested with 30-50 mg/hr of O₃ over a range of distances of about −0.3 to +0.2 inches (e.g. the (−) sign indicates axial orifice 407 recessed in annulus conduit 205 and the (+) indicates that axial orifice 407 extends into reaction chamber 240, 0 indicates that axial orifice 407 is at substantially the same plane as annulus orifice 405). The conditions included a temperature of about 50° C.; a flow rate of NO of about 250 cc/min; a flow rate of O₃ of about 100 cc/min; and at a pressure with reaction chamber 240 of about 200 mm mercury. As illustrated in FIG. 5, a desirable range of distance includes about −0.2″ to about −0.1″, with an optimal distance of about −0.15″. However, as described above a desirable distance depends on a number of factors. For example, depending on conditions, the desirable range of distance may include a distance in a range of about −0.40″ to about +0.10″.

Having described various embodiments and implementations, it should be apparent to those skilled in the relevant art that the foregoing is illustrative only and not limiting, having been presented by way of example only. Many other schemes for distributing functions among the various functional elements of the illustrated embodiments are possible. The functions of any element may be carried out in various ways in alternative embodiments

What is claimed is:

1. An analyzer comprising:
a first conduit configured to channel an annular flow of a first gas;
a second conduit positioned within the first conduit, wherein the outer dimension of the second conduit is separated from an inner dimension of the first conduit by a gap configured to channel an axial flow of a second gas;
a reaction chamber fluidically coupled to the first conduit and the second conduit, wherein the reaction chamber comprises a window on a side opposite from an orifice of the first conduit into the reaction chamber; and
a detector positioned adjacent to a side of the window opposite from the reaction chamber, wherein the detector is configured to receive light produced from a reaction of the first gas and the second gas in the reaction chamber.

2. The analyzer of claim 1, wherein:
an orifice of the second conduit is positioned a distance away from the orifice of the first conduit.

3. The analyzer of claim 2, wherein:
the orifice of the second conduit is positioned in the first conduit to form a mixing region in the first conduit.

4. The analyzer of claim 2, wherein:
the distance of the orifice of the second conduit to the orifice of the first conduit comprises a distance in a range of about −0.40″ to about +0.10″.

5. The analyzer of claim 2, wherein:
the distance from the orifice of the first conduit to the orifice of the second conduit comprises a distance of about −0.15″.

6. The analyzer of claim 1, wherein:
a position of an orifice of the second conduit is adjustable relative to the orifice of the first conduit into the reaction chamber.

7. The analyzer of claim 1, wherein:
the first gas comprises O₃ and the second gas comprises a sample gas.

8. The analyzer of claim 6, wherein:
the sample gas comprises NO.

9. The analyzer of claim 1, wherein:
an internal surface of the reaction chamber comprising the entrance is substantially parabolic.

10. The analyzer of claim 1, wherein:
an internal surface of the reaction chamber comprising the entrance is substantially hemispheric.

11. The analyzer of claim 1, wherein:
the internal surface of the reaction chamber is substantially reflective.

12. The analyzer of claim 1, wherein:
the orifice of the second conduit comprises a nozzle.

13. The analyzer of claim 12, wherein:
the nozzle comprises a flared configuration.

14. The analyzer of claim 12, wherein:
the nozzle comprises a tapered configuration.

15. The analyzer of claim 1, wherein:
the gap comprises a space separation in a range of about 0.005″ to about 0.056″.

16. A method comprising:
(a) channeling an annular flow of a first gas through a first conduit;
(b) channeling an axial flow of a second gas through a second conduit positioned within the first conduit, wherein the outer dimension of the second conduit is separated from an inner dimension of the first conduit by a gap;
(c) reacting the first gas with the second gas to produce light in a reaction chamber fluidically coupled to the first conduit and the second conduit, wherein the reaction chamber comprises a window on a side opposite from an orifice of the first conduit into the reaction chamber; and
a detecting the light produced using a detector positioned adjacent to a side of the window opposite from the reaction chamber.

17. The method of claim 15, wherein:
an orifice of the second conduit is positioned a distance away from the orifice of the first conduit.

18. The method of claim 17, wherein:
the orifice of the second conduit is positioned in the first conduit to form a mixing region in the first conduit.

19. The method of claim 17, wherein:
the distance of the orifice of the second conduit to the orifice of the first conduit comprises a distance in a range of about −0.40″ to about +0.10″.

20. The method of claim 17, wherein:
the distance from the orifice of the first conduit to the orifice of the second conduit comprises a distance of about −0.15″.

21. The method of claim 16 further comprising:
(d) adjusting a position of an orifice of the second conduit relative to the orifice of the first conduit; and
(e) repeating steps (a)-(d) until the position of the orifice of the second conduit produces a maximal value of the light detected from the reaction of the first gas with the second gas.

22. The method of claim 16, wherein:
the first gas comprises O₃ and the second gas comprises a sample gas.

23. The method of claim 16, wherein:
the sample gas comprises NO.

24. The method of claim 16, wherein:
an internal surface of the reaction chamber comprising the entrance is substantially parabolic.

25. The method of claim 16, wherein:
an internal surface of the reaction chamber comprising the entrance is substantially hemispheric.

26. The method of claim 16, wherein:
the internal surface of the reaction chamber is substantially reflective.

27. The method of claim 16, wherein:
the orifice of the second conduit comprises a nozzle.

28. The method of claim 27, wherein:
the nozzle comprises a flared configuration.

29. The method of claim 27, wherein:
the nozzle comprises a tapered configuration.

30. The method of claim 15, wherein:
the gap comprises a space separation in a range of about 0.005″ to about 0.056″.
